(19) United States
(12) Patent Application Publication
(10) Pub. No.: US 2009/0228001 A1
(43) Pub. Date: Sep. 10, 2009

(54) DEVICE AND METHOD FOR THE TREATMENT OF DISEASED TISSUE SUCH AS TUMORS

(75) Inventor: Andrew Pacey, Stevenage (GB)

Correspondence Address: OPPENHEIMER WOLFF & DONNELLY LLP, 45 SOUTH SEVENTH STREET, SUITE 3300, MINNEAPOLIS, MN, 55402, US

(73) Assignee: Emcision Limited, London (GB)

(21) Appl. No.: 11/908,277

(22) PCT Filed: Mar. 9, 2006
(86) PCT No.: PCT/GB2006/000828

§ 371 Date: Apr. 9, 2008

(30) Foreign Application Priority Data
Mar. 10, 2005 (GB) 0504988.7

(51) Int. Cl. A61B 18/18; A61B 18/14; A61B 5/01; A61B 5/053; A61B 5/0215; A61B 8/06

(52) U.S. Cl. 606/33; 606/41; 606/46; 600/549; 600/547; 600/486; 600/454; 606/42

(57) ABSTRACT

A device for the treatment of tumours comprising an elongate catheter (102), a plurality of flexible needles (402) confined within the catheter which, when extended therefrom, take up a curved form and which, together, define a structure for surrounding a tumour to be treated; the needles being arranged to heat and embolise a shell of tissue surrounding the tumour, thereby cutting off the tumour's blood supply. The invention further extends to a method of treatment using such a device.

The present invention relates to a device and method for the treatment of diseased tissue such as tumours, and in particular although not exclusively to tumours within a body of tissue (such as the liver) which will bleed profusely when cut.

When tumours occur within a body of tissue having a heavy blood supply, such as the liver, surgical removal of the tumour by resection has to be undertaken with the greatest of care if significant and potentially life threatening blood loss is to be avoided. Conventionally, liver surgery involving resection is carried out as an open procedure, with the surgeon being required to tie off or to apply localised heating to seal each of the blood vessels within the cut surface. It will be understood that this is a long and difficult procedure, and in recent years other approaches such as ablation have become more popular. In this context, ablation consists of inserting into the centre of the tumour one or more thin needles, and then heating those needles, for example using applied RF energy, to kill the tumour from the inside. Once the tumour has been entirely killed, it can simply be left in place, thereby obviating or all need for resection. A typical prior art device for this purpose is disclosed in U.S. Pat. No. 6,660,002 (Rita Medical Systems Inc).

Unfortunately, there are a number of problems with this approach. First, it is difficult for the surgeon to be sure that all parts of the tumour have been killed. The heating effect of devices such as that shown in U.S. Pat. No. 6,660,002 is non-uniform, and there is a real concern that there may remain small areas of cancerous cells within the tumour that have not been raised to a high enough temperature to kill them. Such areas are most likely to occur adjacent to or within larger blood vessels, since the blood itself will act as a medium for carrying heat away from those areas and thus cooling them. It will be understood that the consequence of leaving in place live cancerous cells which are adjacent to or within a major blood vessel is particularly dangerous, since it is those cells having good blood supplies that are most liable to continue growing, and indeed to continue growing rapidly.

A further disadvantage of the existing device is that it cannot reliably be used on tumours which are larger than about 3 cm in diameter. A tumour which is larger than that cannot easily be heated up throughout its entire volume to a temperature sufficient to kill every part of it. With a large tumour, it may take far too long for the heat to spread from the centre to the outer periphery; and indeed in some cases the outer periphery may never heat up sufficiently to kill the cells at all, particularly in those peripheral areas where heat is constantly being taken away by nearby blood flows. Of course, it is always open to the surgeon to use the device repeatedly, pushing it first into one part of the large tumour, then into another, then into another. However, such repeated use typically takes a large amount of time and also presents the risk that smaller areas may inadvertently be missed between the respective treated volumes.

According to a first aspect of the present invention there is provided a device with the features of claim 1.

According to a second aspect of the invention there is provided a method with the features of claim 28.

Various optional features are set out in the dependent claims.

The present invention finds particular application in preferred embodiments in the minimally invasive removal of deep tumours within highly vascular tissues such as for example the liver, the breast, the bone, the lung, the kidney, the pancreas, the spleen or the uterus. Typically, the device and method will be used in conjunction with a suitable imaging system such as for example ultrasound, x-ray, MRI, or CT.

The shell need not completely surround the tissue or tumour, provided that it is sufficiently extensive to cut off the blood flow to the tissue or tumour to be killed.

The device may consist of a catheter or tube with a control handle at the proximal end and a tissue penetrating distal end. The catheter may have at least an inner lumen, and ideally multiple inner lumens and a double or triple outer wall within the lumens of which are one or two helices of razor-sharp cutting needles which may be diamond section in shape, and which act as RF electrodes or microwave cage, depending upon the energy source being supplied. The particular device may be chosen from a range of sizes of such devices following verification of the tissue volume to be removed using appropriate imaging.

The needles may be slidable within the device and may be pre-stressed or made of a memory metal such as Nitinol and guided by ceramic or polymer formers such that they form one or two teardrop shapes around the tissue to be removed when extended from the distal end of the catheter; typically, a larger and a smaller teardrop shape with the needles generally parallel to each other and approximately 1 cm apart (i.e. the smaller teardrop has a diameter 2 cm smaller than the larger one).

The placing of the needles may be verified by imaging and the tissue surrounding the tumour then irradiated with electromagnetic radiation of RF or microwave frequency, causing the collagen surrounding the blood vessels to constrict and the blood to coagulate. Having created a plane of avascular tissue, the target tissue to be removed (such as a tumour) may be safely removed by a corer and rotating cutter placed in a central lumen of the device without risk of causing further metastases. The target tissue itself need not be ablated.

The rotating cutter may be formed from cutting blades which are contained within or inserted down the primary lumen of the device and expanded in operation to create a substantially spherical cutter whose diameter is slightly less than the cage created by the needle array. Tissue cut by this method may then be removed by flushing a physiological solution into the volume created by the spherical cutter and aspirating the contents. All of the target tissue can be removed and the cutters can then be removed and the remaining void inspected by optical means to ensure that the tumour is surrounded by a plane of avascular tissue. Alternatively, the cutter may not be substantially spherical but may take on any suitable form to cut the tissue.

By choosing the correct size of device, a minimum amount of healthy tissue can be removed around the tumour or other tissue to be removed.

In one preferred apparatus and method the tumour is surrounded by a cylinder, consisting of a circular array of substantially straight needles, with a central needle/probe. The straight needles are easy to manufacture. The tumour can be considered to be in a cylinder consisting of two discs separated by a cylindrical circumference, and the cylinder surface is heated in two or three stages, first the circumference, then a lower and an upper disc at either end of the cylinder. The cylindrical circumference is then heated by connecting adjacent needles to alternate polarity, either in series in successive pairs, or in parallel by simultaneously connecting the alternate needles together.

Following this the upper and lower disc are heated separately or simultaneously. The central needle is insulated apart from one or two short sections. The lower disc is heated by connecting the central needle to one polarity, and one or all of the outer needles to the other polarity. The un-insulated section is positioned at the bottom of the heated zone. The upper disc is heated in the same way, either by translating the central needle so that the uninsulated section is positioned at the top of the heated zone, or by having a second uninsulated section on the central needle.

Finally the inside of the tumour may be heated by stepping the uninsulated portion of the central needle through the zone from the upper disc to the other. Alternatively there may be an additional (third) uninsulated section on the central needle, that can be individually switched. This uninsulated section will extend from the lower disc (and its section) to the upper disc.

The device and method of the present invention allows surgeons to operate safely and efficiently on larger tumours than has previously been possible, and in particular on such tumours which may occur within highly vascular tissue.

Although the described embodiments of the invention are particularly useful in conjunction with laparoscopic procedures, it will be understood that in its most general form the invention may also find application in open surgical procedures.

The device and method may be used by a surgeon or a radiologist by way of a laparoscopic or an open procedure, under general or local anaesthetic.

The invention may be carried into practice in a number of ways and a variety of different embodiments will now be described, by way of example, with reference to the accompanying drawings, in which:

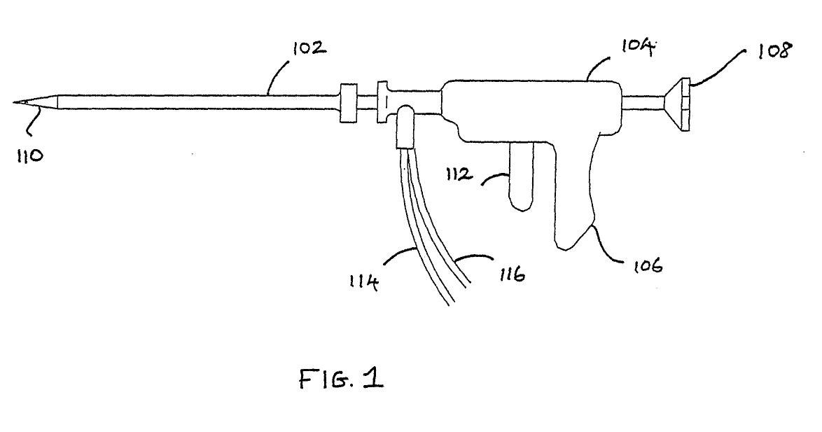

FIG. 1 shows a laparoscope suitable for use with the embodiment of the present invention;

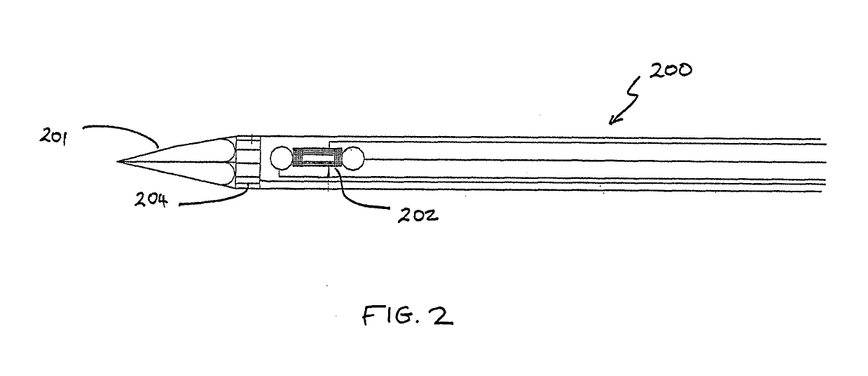

FIG. 2 shows a core probe for insertion into the laparoscope of FIG. 1;

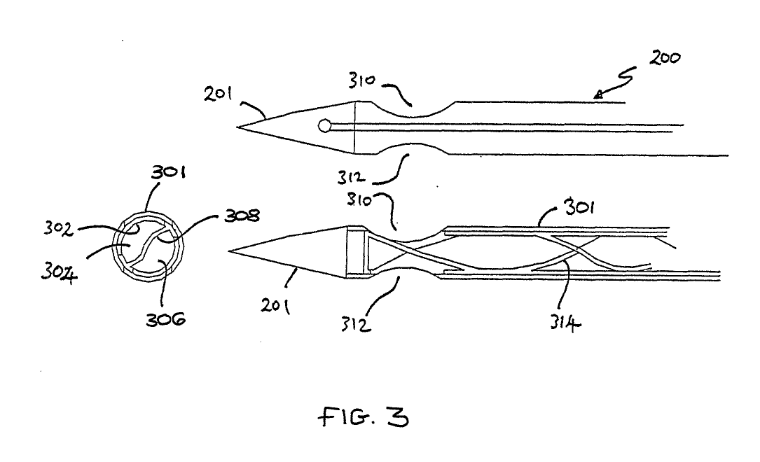

FIG. 3 illustrates the internal structure of the core probe;

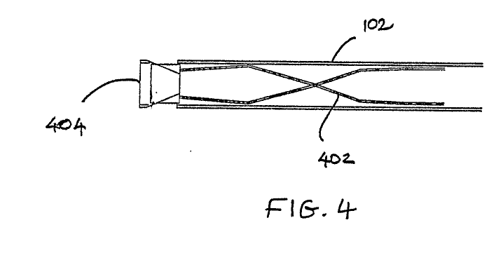

FIG. 4 shows the pre-stressed needles, as contained within the body of the core probe;

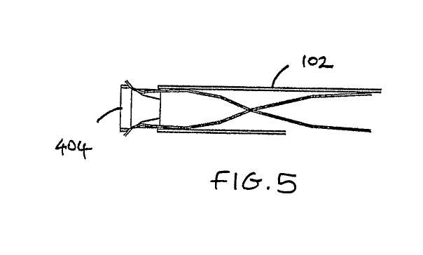

FIG. 5 shows how a needle guide helps guide the needles as they are extended from the probe;

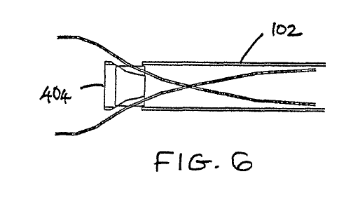

FIG. 6 shows the needles starting to take up their preformed shapes;

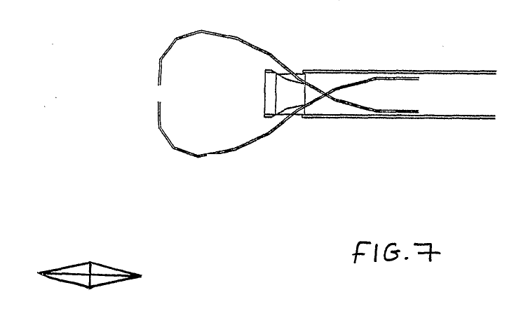

FIG. 7 shows the needles fully extended, and forming a cage;

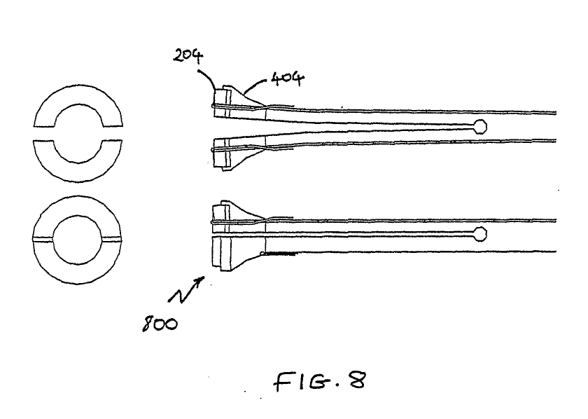

FIG. 8 shows a blood flow Doppler;

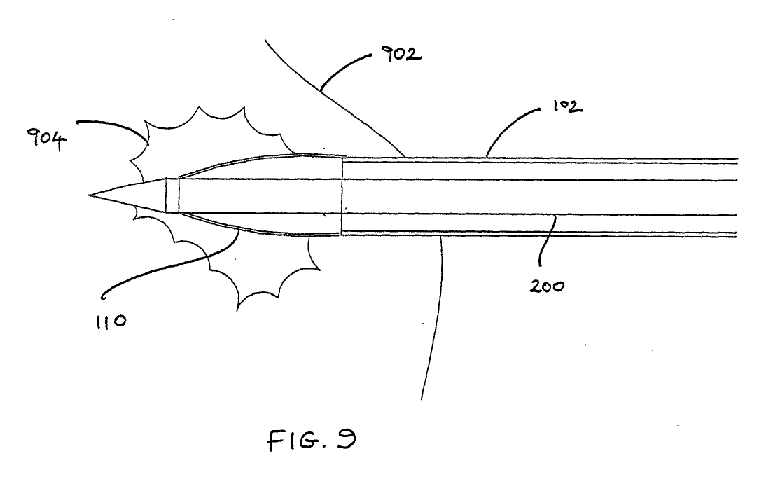

FIG. 9 illustrates a first stage of the procedure, in which the probe is inserted into the tissue;

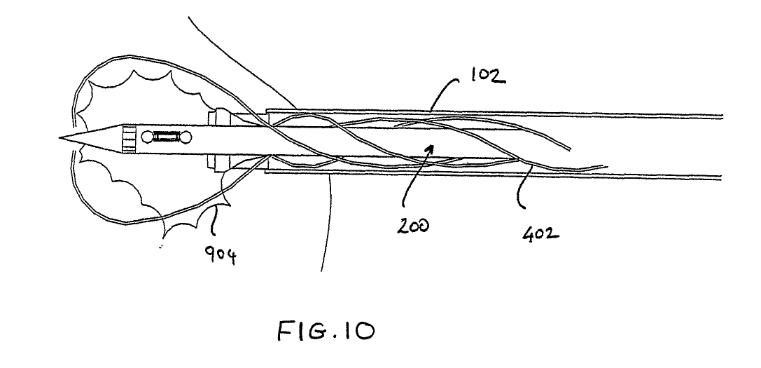

FIG. 10 shows the insertion cover removed, and the needles in their operational position;

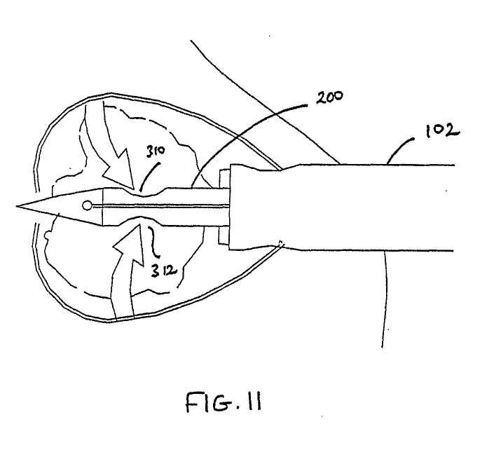

FIG. 11 shows the tumour being aspirated;

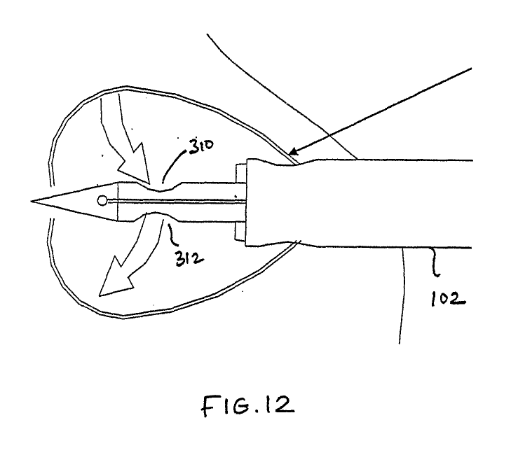

FIG. 12 illustrates an alternative arrangement in which one lumen of the probe is used for aspiration, with the other being used to supply liquid to the treatment area;

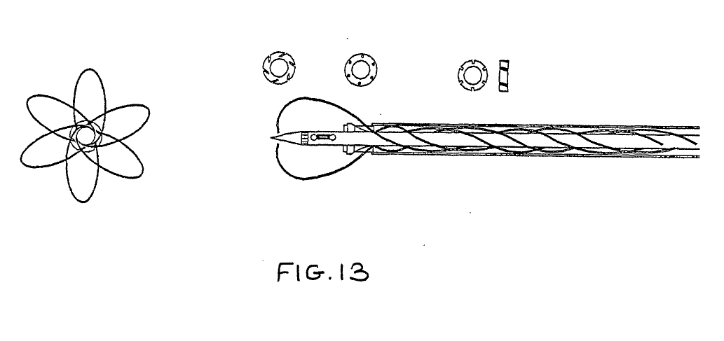

FIG. 13 shows an embodiment in which the probe has six needles;

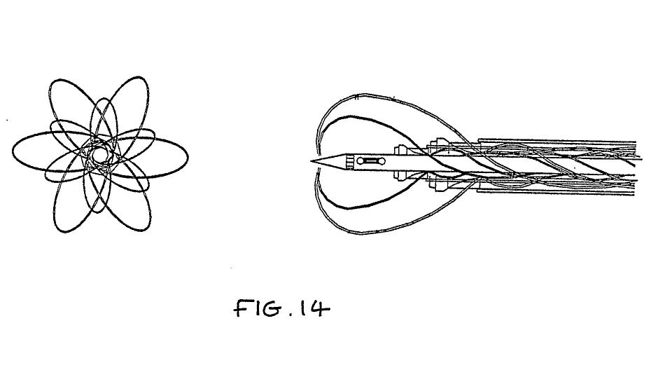

FIG. 14 shows an alternative embodiment having two separate sets of needles, each having a different radius;

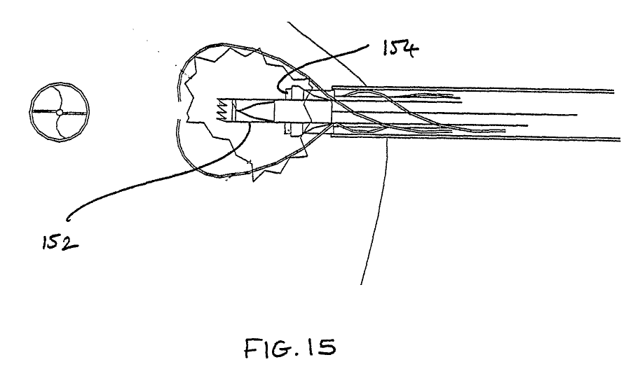

FIG. 15 shows the system with the probe having been removed and replaced with a coring cutter;

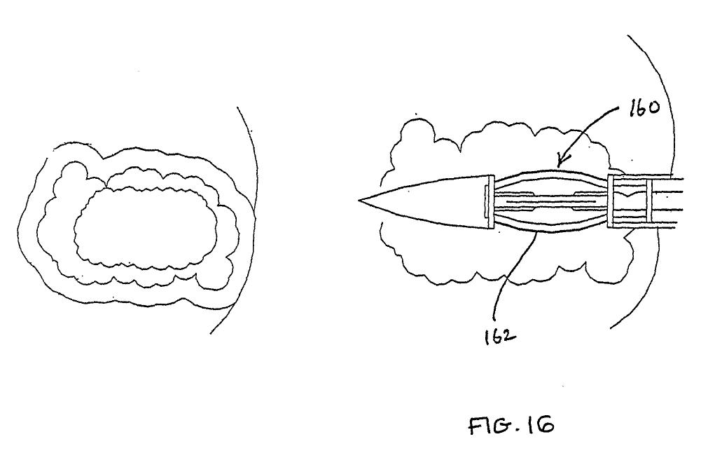

FIG. 16 shows a rotating cutter, suitable for removing larger volumes;

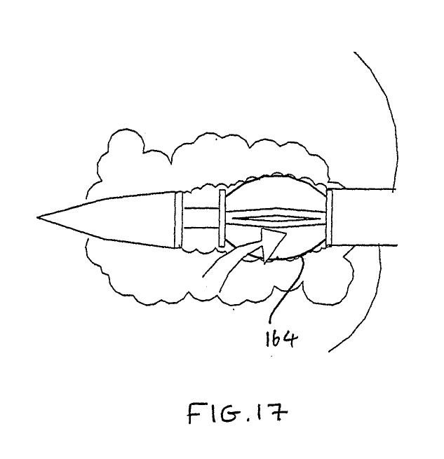

FIG. 17 shows the rotating cutter in operation;

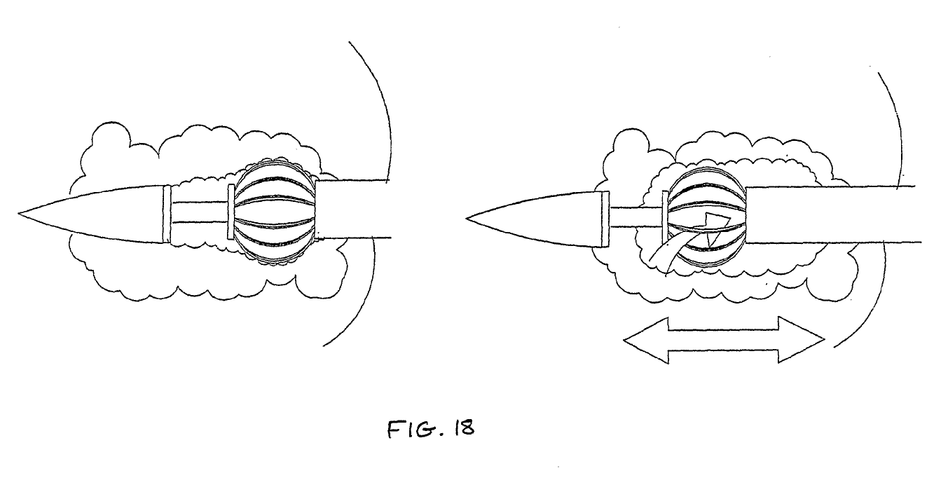

FIG. 18 shows the way in which the rotating cutter can be traversed;

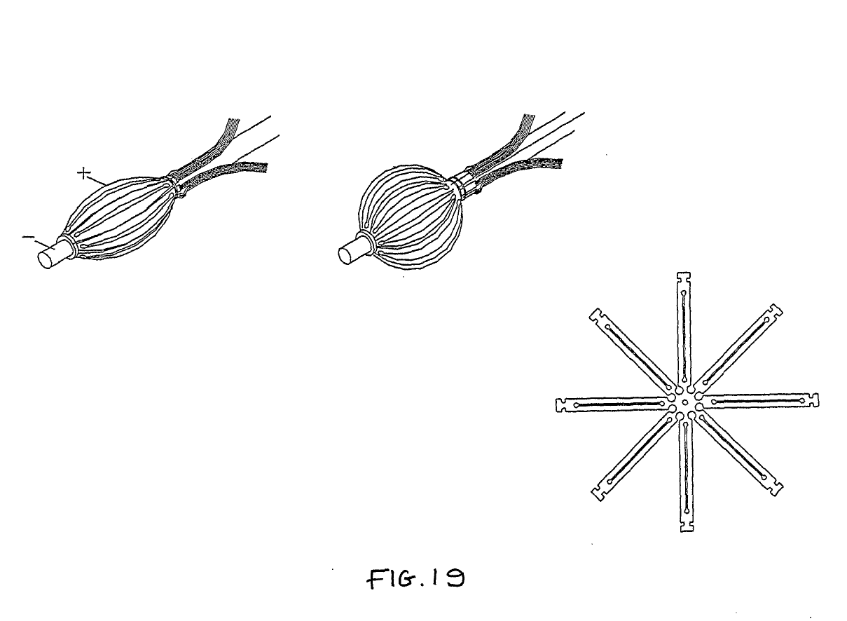

FIG. 19 shows in more detail the blades of the rotating cutter;

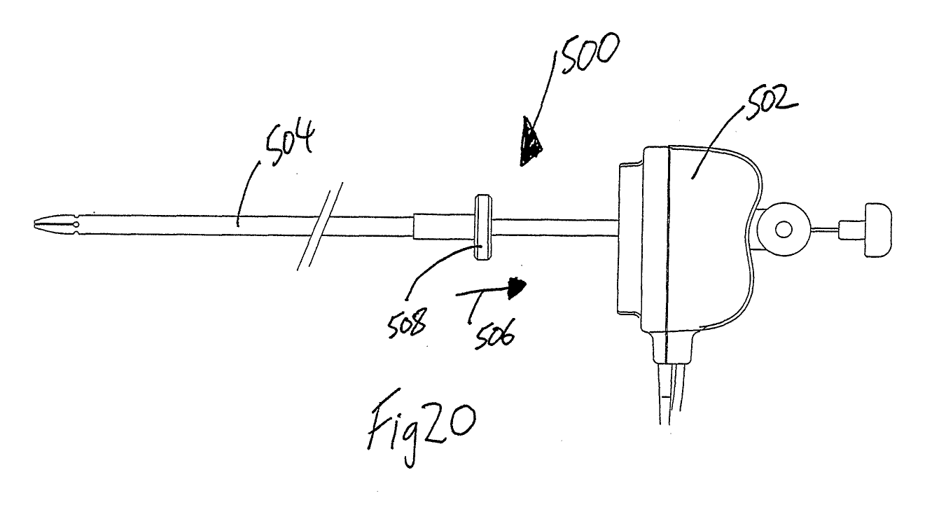

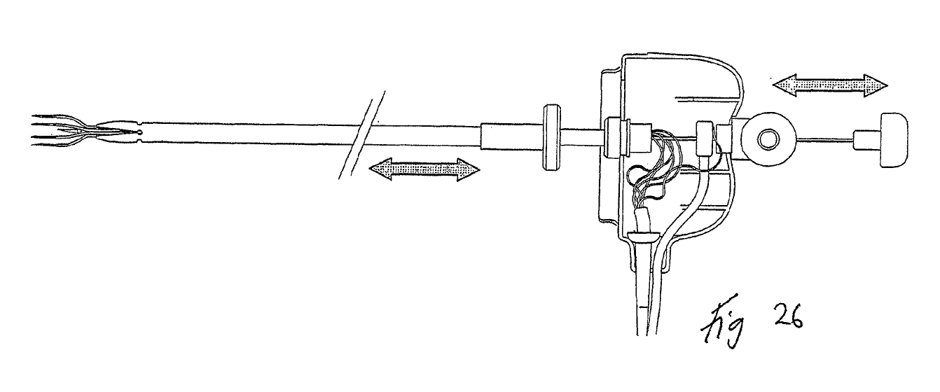

and FIGS. 20 to 28 show an alternative embodiment of a device in accordance with an embodiment of the invention as well as methods of treatment using said device.

FIG. 1 shows a laparoscope suitable for use with the various embodiments of the present invention shown in FIGS. 1 to 19. The laparoscope has a hollow laparoscope catheter 102, for insertion into a body of tissue to be treated, and a laparoscope body 104 with a handle 106. An insertion port 108 allows the surgeon to insert probes as required, through the laparoscope and along the hollow catheter 102. The catheter itself has a cover 110, over its distal end, which can be retracted by means of a trigger 112, to expose the operational end of the probe. Fluid in and out lines, 114, 116 are provided for supplying fluid to and for aspirating from the volume of tissue being treated.

FIG. 2 is an external view of a core probe 200, suitable for insertion within the catheter 102 of FIG. 1, according to an embodiment of the present invention. The probe 200 has a sharpened distal end 201 and, adjacent the distal end, an optional sensor array 202 and a blood flow Doppler 204 (also optional and to be described in more detail below, with reference to FIG. 8). The sensor array 202 comprises a small chip which has on-board sensors for a variety of diagnostics, including blood pressure, impedance and temperature. The measurements may be transmitted back along the probe for external real time analysis, or alternatively the chip itself may include some calculation and analysis capabilities, with only the results of the analysis being fed back out.

The structure of the probe 200 is shown in more detail in FIG. 3. The probe has a rigid cylindrical body 301 and a pointed end 201, as previously mentioned. Near the distal end are a pair of opposing apertures 310, 312. Contained within the body is a disposable sleeve of a plastics material, comprising an outer cylindrical wall 302 and a longitudinally extending central wall 308 which separates two longitudinal channels or lumens 304, 306. The external wall of the sleeve has a moulded spiral 314 which aligns with cut outs (not shown) in the cylindrical body 301. When in position, the aperture 310 allows access to the lumen 304, and the aperture 312 allows access to the lumen 306. The disposable sleeve allows for the cleaning and servicing of the rest of the probe, which is re-usable.

As is best shown in overview in FIG. 10, the annular space between the catheter 102, (shown in FIG. 1) and the probe 200 (shown in FIGS. 2 and 3) contains a plurality of pre-stressed needles 402 in a spiral formation.

FIG. 4 shows the catheter 102 and the needles 402, with the probe 200 being omitted for clarity. As may be seen in FIG. 4, at the distal end of the catheter 102 is a ceramic or polymer needle guide 404. When the laparoscope trigger 112 is depressed the needles are urged forwardly and extend out of the end of the catheter 102, their emergence being assisted by the guide 404 as shown in FIG. 5.

As shown in FIG. 6, once the needles have left the catheter 102, they will start to take up their pre-formed radius, eventually reaching the position shown in FIG. 7, where the needles together define a tear shaped cage which will, in use, enclose the tissue volume destined for treatment. That will usually be a tumour. Instead of being curved, the needles may be angular in form—i.e. each may consist of a series of straight or curved sections coupled by a tightly curved or angular section (not shown). The cage shape could also be generally spherical or cylindrical.

FIG. 7 also shows that the needles have a diamond shaped section, with razor edges and sharpened tips, to aid their passage through the tissue. The spiral configuration of the needles causes them to twist as they move forward, so increasing cutting efficiency. The needles may be manufactured either of a memory metal such as Nitinol, or alternatively may be pre-stressed to ensure that they take up the required shape once they have been freed from the catheter.

The needle guide 404 is shown in more detail in FIG. 8. The guide 404 forms the rear portion of a blood flow Doppler unit 800 which includes a blood flow sensor or PZT 204. The PZT may also be seen in FIG. 2.

In operation, a probe 200 is first inserted into the catheter 102 via the insertion port 108, as shown in FIG. 1. The catheter is then inserted (typically via the abdominal wall) into an organ such as the liver 902. The probe is moved into the organ guided by ultrasound, doppler, or any other available imaging technique until the desired location is reached. The trigger 112 is then depressed to retract the insertion cover 110 and to push the needles forward as shown in FIG. 10. As the needles move forward, they twist, forming a cage which surrounds a tumour or other tissue area 904 to be treated.

Once the device is in place, RF or EM energy is supplied to two adjacent needles within the cage, causing heating and embolisation of the segment of tissue between the two needles. Once the tissue has been heated to a sufficiently high temperature for a sufficient time to embolise any blood vessels, the RF power is switched off and is re-applied between the adjoining pair of adjacent needles. This process is repeated so that, segment by segment, the entirety of the peripheral tissue which surrounds the tumour 904 is embolised. The process may be automated, and carried out under computer control.

It will be understood that there are of course many ways in which the tissue adjacent to the needles can be heated sufficiently to cause embolisation. If sufficient power is available, all of the individual segments between the needles could be heated up at once. Where RF energy is to be used the individual needles may be either monopolar or bipolar. Alternatively, microwave energy may be used. A microwave generator (not shown) may be provided as part of the probe or the catheter, or alternatively externally generated microwave energy may be guided along a wave guide within or formed by the catheter. The needles themselves can be microwave sources, i.e. dipoles.

Once the blood flow has been entirely occluded within the peripheral area defined by the cage, any tumour 904 must then die for lack of a blood supply. Of course, where the tumour lies at or near the surface of an organ, the blood flow to the tumour may be occluded by partially surrounding the tumour. It is not always essential to embolise a complete shell of surrounding tissue, but rather the important thing is to block the blood flow to the tumour or other volume of tissue to be killed. Surface or near-surface tumours could be approached either from above the surface in question or from below (i.e. through the organ).

In some situation, the tumour can simply be left in place without resection being necessary. In such cases, it may be desirable to dehydrate the body of the tumour to prevent infection. If the peripheral heating caused by the needles has been sufficient to drive out water from the entire body of the tumour, no further treatment may be necessary. However, if the tumour is particularly large or if for any other reason the surgeon cannot be sure that it has heated through sufficiently to drive off the water, an additional desiccation step may be necessary. Heating the centre of the tumour could be carried out either by providing suitable RF electrodes (not shown) on the probe itself, or alternatively by the repeated use of a conventional heating probe inserted as necessary within the body of the tumour, once the cage has been retracted. Repeated use of a spot heating probe does not necessarily take very long, since the time taken to heat a tumour sufficiently to drive off water is much less then would be required in a conventional treatment regime actually to kill the tumour cells.

In some circumstances it may be desirable or necessary to remove the body of the tumour. That can easily be achieved with the present invention, in a number of different ways, to be described below.

Where the tumour is of a suitable size, the heating effect caused by the needles may be enough not only to embolise the region surrounding the tumour, but also slightly to soften the body of the tumour itself. As best seen in FIG. 11, as the tissue of the tumour softens and dehydrates, it is sucked into the probe 200 via the apertures 310, 312. The aspiration will advantageously provide a negative pressure inside the heated zone, preventing the undesirable egress of tumour material out of the heated zone. The resultant tissue slurry may be extracted using standard aspiration equipment (not shown). Sensors (not shown) or the core and/or the needles may be provided to measure the force needed to deploy the device, thereby providing an indirect measure of tissue softening.

An alternative possibility, shown in FIG. 12, is to use one of the lumens for aspiration via the aperture 310, and to use the other as a flushing channel or to deliver a drug or gel to the treatment area via the second aperture 312.

An alternative probe embodiment is shown in FIG. 13. In this embodiment, six needles are provided, all of which are similar and follow the same helix within the canula. A ceramic needle guide or former is preferably provided, shaped as shown in the diagram. Such an arrangement is convenient for diathermy use. The embodiment of FIG. 13 is also useful for microwave applications, in which the needles together define a focusing cage.

Another possible embodiment is shown in FIG. 14, in which a double set of needles is provided, with one set being pre-stressed to define an inner cage and the other set pre-stressed to define an outer, slightly larger cage. Such an arrangement may be useful for the treatment of larger tissue areas and/or for ensuring that a hollow shell is created thick enough to close significant arteries. Where RF power is to be supplied, there are a variety of different needle wiring arrangements that could be used. One possibility is for the needles to be alternating positive and negative around each of the two cages, with each cage operating independently of the other. Provided that the cages are sufficiently close together, the respective embolised peripheries will merge into one thicker periphery. Alternatively, it would be possible for each electrode in the outer cage to have an opposite polarity from a corresponding electrode in the inner cage. RF power could then be applied between the respective inner and outer electrodes, to embolise the space between. The two cages may also be separately energizable.

In this and other double caged embodiments, embolisation of the tissue between the inner and outer cages could alternatively be effected by propagating microwave energy into the space between the two cages. By suitably choosing the spacing of the inner and outer needles, the microwave heating effects can be contained largely within the desired peripheral shell.

One possibility (which is applicable to all the described embodiments) is for the needles all to be wired as positive RF electrodes, with the probe itself forming the negative electrode, or visa versa.

There are some circumstances where it may be desirable for the surgeon to remove the tumour, either in whole or in part, once its peripheral blood flow has been cut off as previously described. One way of doing that is shown in FIG. 15, in which the probe has been removed and replaced with a coring cutter 152 to remove the treated tissue. If required, the cutter may incorporate dual lumens (not shown) allowing both aspiration of the cut tissue, and also flushing and/or delivery of drugs to the treated area. The forward facing doppler 154 ensures that blood flow has stopped prior to coring.

An alternative cutting arrangement, this time using a rotating cutter 160 is shown in FIG. 16. This cutter incorporates flexible blades which gradually expand about the central axis as cutting proceeds. This is best shown in FIG. 17, which also illustrates the tissue being removed by the aspirator. Flushing may also be used to assist removal. FIG. 16 also shows how, after cutting, a shell of dead tissue remains, surrounded by the shell of embolised or coagulated tissue.

As shown in FIG. 18, larger cutting areas may be obtained by traversing the blades along the longitudinal axis of the catheter. Finally, FIG. 19 shows the manufacturing patterns for the rotating cutter blades. The blades are typically fabricated out of sheet material, and then sharpened prior to assembly. The front face or the entirety of each blade may be coated to stop sticking. As also shown in FIG. 19, the cutting blades may if desired themselves act as electrodes for supplying RF energy to the tissue to be treated. As shown in that Figure, the blades may have one polarity, with the core of the cutter having the opposite polarity. The distal or the opposite ends of the needles may, in some embodiments, be selectively coated with a non-conductive coating.

It will be understood that there are a variety of other mechanisms by which a volume of tissue such as a tumour, once treated, may be resected. In addition to the embodiments of FIGS. 15 to 19, conventional macerators or other resecting devices could be used.

FIG. 1 shows a core probe enabling a system of electro surgical instruments to work from a common laparoscopic platform. FIG. 2 shows the end of the core probe which has integrated mems for tissue diagnostics. Luer connections and Y junction allow for the switching of channels between needles in use. The needles showing in FIG. 4 are pre-stressed so that they form an enclosure when fully extended, needle guide 404 being ceramic and helping the path of the needles. As shown in FIG. 5, the needle guide 404 pushes the needles outwardly as they are deployed, helping them to take on their pre-formed radius or curvature. As shown in FIG. 6, once the needles have left the probe case they will start to take the pre-formed radius/curvature/non-linear shape. As shown in FIG. 7, once fully deployed the needles for a “cage” which encloses an area for treatment. The needles cut as they travel to form the curved envelope shown in FIG. 7. The pzt (piezo-electric transducer) allows for forwarding facing doppler to monitor blood flow prior to cutting.

As shown in FIG. 9, the probe is inserted into the tissue to be treated. Ultrasound or doppler can be used to assist location. As shown in FIG. 10, the insertion cover is pulled back and the needles pushed forward, twisting as they travel forming an enclosure. Tissue condition is monitored during treatment to allow the correct amount of electro magnetic power to be applied for optimum ablation of tissue. As shown in FIG. 11, as tissue is softened, it is sucked into the inner core probe via an aspirator, the tissue slurry being vacuumed using standard aspiration equipment (not shown). The probe core has two channels and it is possible to use one channel/lumen as a vacuum for aspiration and the other as a flushing channel or to deliver drugs/gel to the treatment area. A doppler is provided to confirm if blood flow is present.

As shown in FIG. 13, using ceramic forms it is possible to have 6 needles all following the same helix, thereby allowing all needles to be positive charged with the main probe being negative charged for diathermide use. The configuration shown in FIG. 13 may also consist of a focusing cage or microwave EM energy application. With a double layer of needles, as shown in FIG. 14, two helix set of needles are provided, each set having a different radius or size such that the two sets do not touch one another. This may allow the treatment of larger tissue areas and for ensuring that a hollow shell is created thick enough to close significant arteries. As shown in FIG. 15 the forward facing doppler 154 shows that blood flow has stopped prior to coring and the probe is removed and replaced with a coring cutter to remove the treated tissue. Vacuum aspiration and flushing are possible.

FIG. 16 shows a rotating cutting concept, similar to coring, but this may be used to remove larger areas/volumes of tissue, the probe being insertable into a tumour for tissue coagulation, the cutting blade electrodes then being rotated. As shown in FIG. 17, as the blades open, the cut tissue may be removed by an aspirator. Flushing can also be used to assist removal. Traversing the blades as shown in FIG. 18 allows for large de-bulking of the tissue. As shown in FIG. 19, the blades may be fabricated out of sheet material and then sharpened prior to assembly and the blade and core may act as electrodes, the front face of the blade being coated for insulation to stop sticking of tissue thereto.

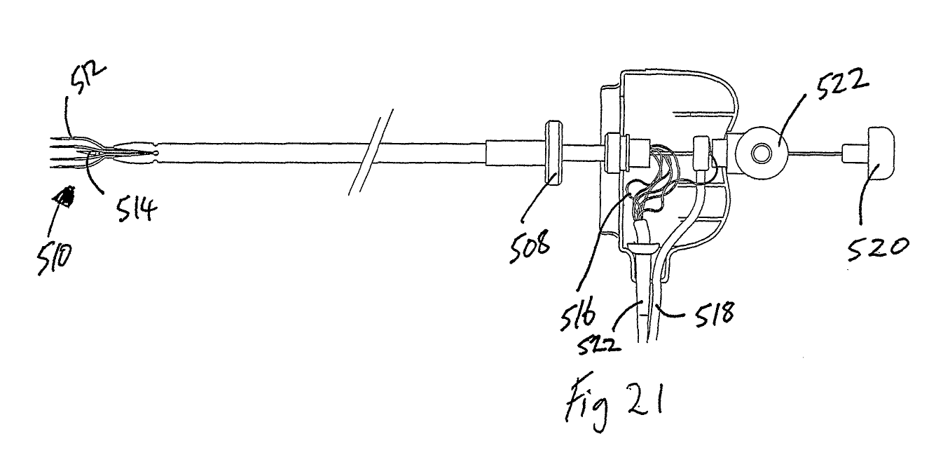

In the embodiment of FIGS. 20 to 27, a percutaneous or laparoscopic device 500 includes a main body 502 and a slidable cover 504 which may be slid from the configuration shown in FIG. 20 in the direction of arrow 506 by pulling on the knob 508 in order to expose and deploy needle array 510 as shown in FIG. 21. Needle array 510 includes 6 outer needles 512 arranged in a cylindrical pattern around a central needle 514, the needles being arranged for application of electromagnetic energy to tissue such as RF tissue via conductor cables 516. An aspiration tube 518 is also provided for providing aspiration through hollow central needle 514 as we described below. The hollow aspiration needle 514 may be slid axially relative to outer needles 512, using knob 520 and may be locked in position using lock wheel 522. Tube 518 and electrical cable assembly 522 may be connected to standard aspiration equipment and switching equipment (not shown) for driving the electrodes, 512, 514 with electromagnetic power. The knob 520 may be used to change the “cook” zone/ablation zone for tissue treated by the needles, 512, 514 by sliding the central needle 514.

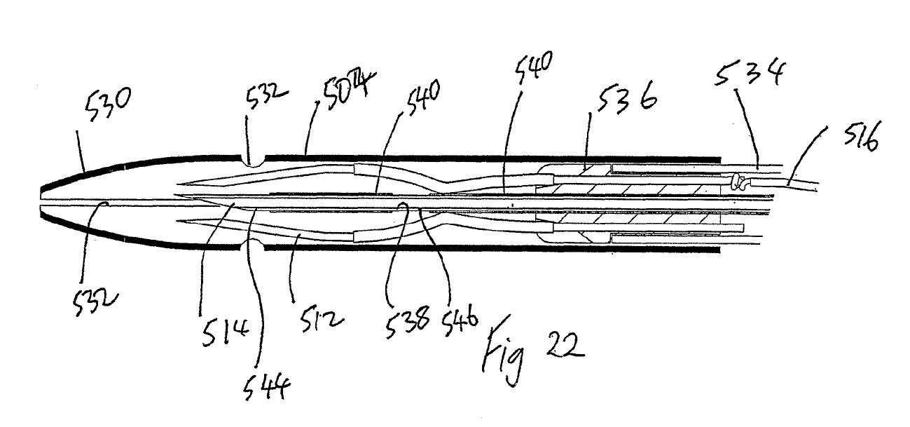

As FIG. 22 shows, the plastic outer cover 504 includes a sharpened tip 530 for piercing tissue. The tip is expandable by virtue of 4 slots 532 formed therein. The needles 512, 514 are mounted to a stainless steel main shaft 534 by a PEEK needle hub 536. Needles 512, 514 are connected to the conductors 516. Central needle 514 is a “hypo” needle due to its central lumen 538 for aspirating as will be described below. Needle 514 may include 2 lumen/channels in order to allow for aspiration and flushing simultaneously. Therefore needle 514 includes insulated portions 540 insulted by standard insulated materials such as polyisocyanate materials. Central needle 514 also includes exposed conducting end 544 which is sharply tapered and axially spaced conducting portion 546.

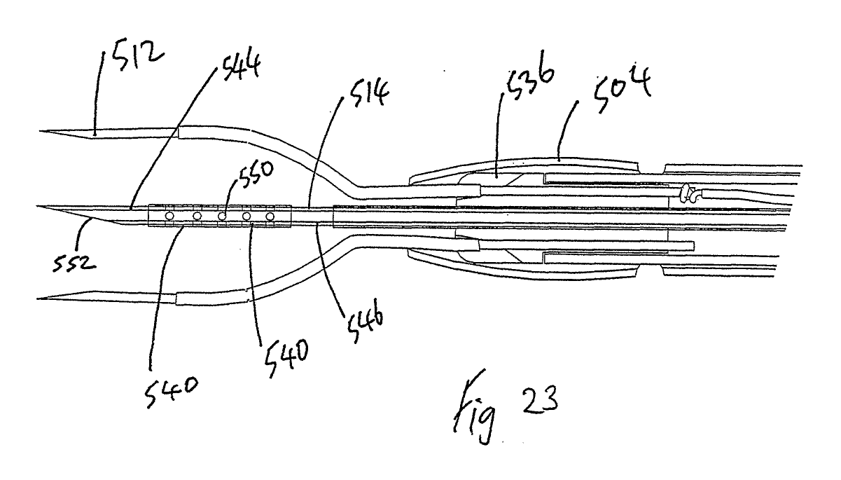

As shown in FIG. 23, once the cover is pulled by the knob 508 to deploy the needles to the FIG. 21 configuration, during which process cover 504 hinges over needle hub 536, axially slidable central needle 514 may be slid between retracted configurations shown in FIG. 21 and extended configuration shown in FIG. 23 with the needles 512, 514 located in tissue and electromagnetic power applied to cables 516 at appropriate frequency and voltage, cook or ablation zones may be formed in the tissue adjacent conducting portions of splayed outer needles 512 and conducting portions 544, 546 or central needle 514. Insulated portion 540 of central needle 514 includes a series of aspiration ports 550 allowing aspiration therethrough as well as through the aspiration port 552 at the tip of the needle 514. The insulation prevents sticking of tissue in a region of the ports 550.

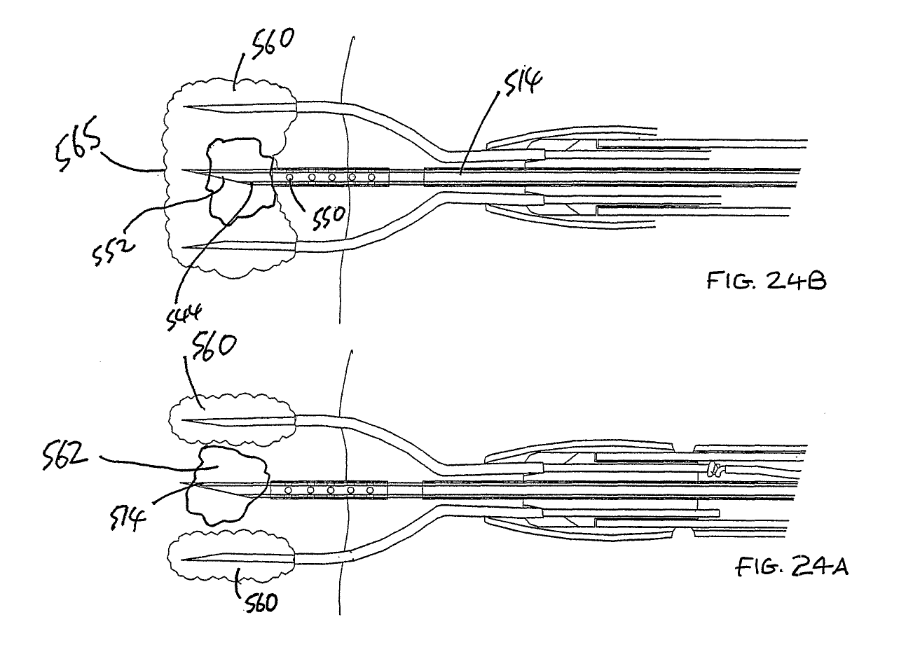

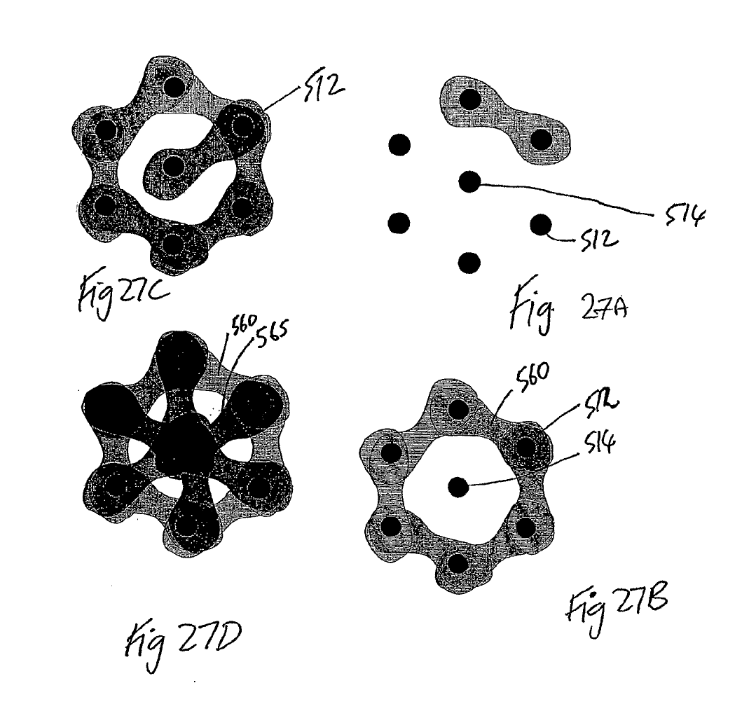

FIG. 24a shows that in a first step, a ring-shaped heating/ablation zone 560 may be formed in the tissue by switching between outer needles 512 with alternate needles being provided with opposite charges and no charge on central needle 514. Central needle 514 may be deployed from retracted position shown in FIG. 21 to extended position shown in FIG. 24a before or after this step occurs. Accordingly, tumour 562 in tissue is surrounded by cylindrical ablated zone 560. This step in the process may be seen axially in FIGS. 27a and 27b and, as will be seen, in these Figures, different circumferential portions of cylindrical ablation 560 may be formed in stages between pairs of outer needles 512 if desired, rather than ablating the entire circumferential ring at the same time.

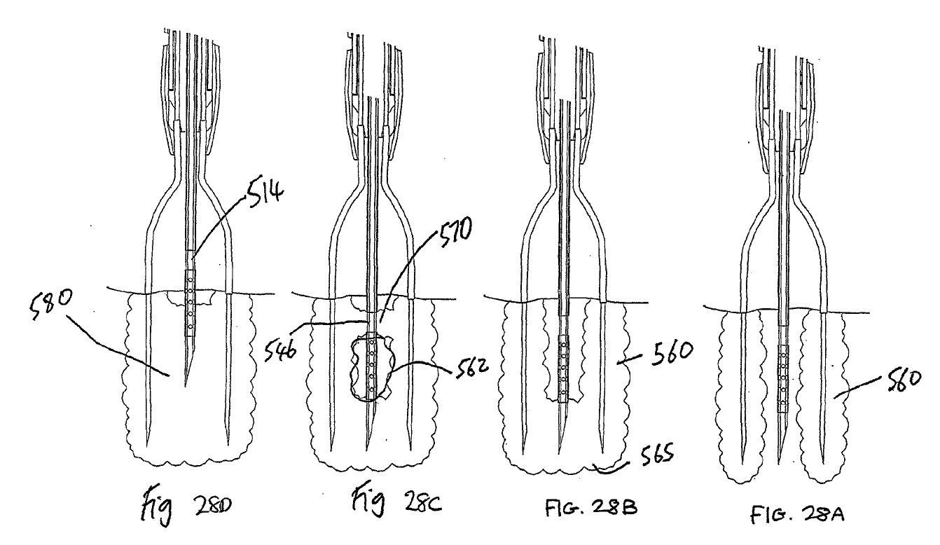

Next, as shown in FIG. 24b and FIGS. 27c and d, conducting portion 544 of central needle 514 at the tip of the needle may be charged positive or negative and outer needles 512 may be provided with opposite polarity to form an end cap 565 on one end of cylindrical ablation zone 560 and, optionally, aspiration through aperatures 552, 550 may commence at this point. In a similar way, FIGS. 28a and 28b show equivalent steps to those in FIGS. 24a and 24b. The process may be continued in FIG. 28c by forming a second disk or end cap 570 by passing voltage to second conductor portion 546 of central needle 514 in order to surround tumour 562 with ablated tissue to cut off any blood supply thereto so that tumour will be killed. As shown in FIG. 28d central needle 514 may be retracted while electromagnetic power is applied thereto in order to ablate the central core of ablation zone 580, if required. FIG. 27d shows an end view of the ablations zones in FIG. 28b configuration. It will be appreciated that the main ablation areas as depicted in FIG. 27 are schematic and that in practice no gaps will be left between zones of ablation 565 such that tumour 562 is fully isolated.

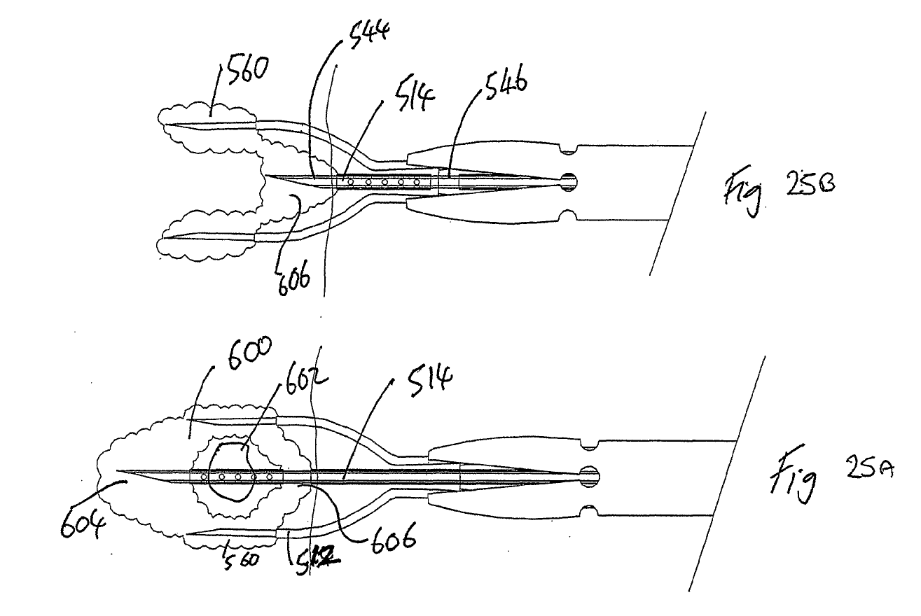

FIG. 25a shows how a hollow ablated zone 600 may be formed around a tumour 602 by further extending aspiration needle 514 relative to outer needles 514 such that 2 end regions or disks 604, 606 may be formed at either end of the cylindrical ablation zone 560 to isolate tumour 602 and tumour 602 may be aspirated with needles 512, 514 in this configuration. As shown in FIG. 25, if central needle 514 is fully retracted, only one electrode ring 544 namely at the tip of needle 514 may transmit electromagnetic power to tissue with spaced second ring 546 being isolated from tissue, such that, if desired by the surgeon, an ablation configuration with a cylindrical sidewalk and one end disk 606 may be provided.

It will be appreciated therefore that central needle is advantageous in that it allows lids or disks or end caps to be formed on cylindrical operated volume of the tissue. If the first RF/EM power application step is made with the outer needles 512 with the inner central needle 514 refracted, then bleeding may be avoided when the central needle is extended. As shown in the drawings, six outer needles may be provided although other numbers of outer needles such as eight outer needles are envisaged.

The typical size of embodiments according to the present invention will vary according to the required application, but where physiologically possible it is preferable, for the sake of safety, for the depth of the embolised shell which surrounds the tumour to be at least 1 cm. Where that is undesirable or impossible because of adjacent structures, the thickness of the shell can be reduced to as little as 1 mm.

1. A device for the treatment of tumors comprising an elongate catheter, a plurality of needles confined within the catheter which, when deployed therefrom, together, define a structure for surrounding a tumor to be treated; the needles being operable to heat and embolize a shell of tissue surrounding the tumor, thereby cutting off a blood supply of the tumor.

2. A device as claimed in claim 1 in which the needles are flexible.

3. A device as claimed in claim 1 in which the needles are arranged, when extended, to take up a curved or angular form.

4. A device as claimed in claim 1 in which the needles are arranged to act as electrodes to which RF power is applied to heat the said shell of tissue.

5. A device as claimed in claim 4 in which adjacent needles within the structure are arranged as electrodes operable with opposing polarities.

6. A device as claimed in claim 1 including an elongate probe which extends along a longitudinal axis of the catheter and which includes a measuring device for measuring a characteristic of the tissue within the cage.

7. A device as claimed in claim 4 including an elongate probe which extends along a longitudinal axis of the catheter, the needles of the structure being arranged as electrodes having a different polarity from that of the probe.

8. A device as claimed in claim 6 in which the measuring device comprises a blood pressure sensor, an impedance sensor or a temperature sensor.

9. A device as claimed in claim 1 including a blood flow doppler for measurement of blood flows within the tissue inside the cage.

10. A device as claimed in claim 1 including a lumen for aspiration of at least some of the tissue within the cage.

11. A device as claimed in claim 1 including a cutter for cutting tissue in the region of the tumor.

12. A device as claimed in claim 10 including a cutter for cutting the tissue to be aspirated.

13. A device as claimed in any claim 1 including a first plurality of needles which define a first structure and a second plurality which define a second, smaller structure within the first structure.

14. A device as claimed in claim 13 in which the needles are arranged to heat and embolize a volume of tissue between the inner and outer structures.

15. A device as claimed in claim 1 which is arranged to embolize the shell as well as the tumor by the application of microwave energy within the structure.

16. A device as claimed in claim 14 in which the said volume of tissue is embolized by the application of microwave energy or RF energy between the inner and outer structures.

17. A device as claimed in claim 1 in which the needles are arranged to form an array having a central electrode needle and a series of outer needles spaced about the central needle; the outer needles preferably being regularly spaced about the central needle, preferably having operative end portions configured in a straight-sided cylindrical array.

18. A device as claimed in claim 17 in which the central needle is movable relative to the outer needles, preferably axially slidable relative thereto along a longitudinal direction of the device.

19. A device as claimed in claim 17 in which the central needle includes one or more lumens for aspiration and/or flushing.

20. A device as claimed in claim 17 in which the central needle includes axially spaced conducting portions for applying electromagnetic energy to tissue; preferably in which the conducting portions are individually activatable by electromagnetic power source.

21. A device as claimed in claim 20 in which the conductive portions are spaced apart by an axially extending insulator portion in which the axially extending insulator portion includes one or more apertures therein for aspiration and/or flushing.

22. A device as claimed in claim 17 which the outer needles have straight conducting portions adjacent the ends thereof and the central needle is straight, the straight conducting portions being parallel to the central needle.

23. A device as claimed in claim 17 which includes a needle cover, the cover having a tissue-piercing tip, the cover being slidable relative to a main body of the device to expose the needles; and preferably in which the tip is expandable, preferably including a series of expansion slots, to enable deployment and expansion of the needles therethrough from a packed, covered configuration to an expanded operative configuration.

24. A device as claimed in claim 17 including a switching system for switching EM power between the needles.

25. A device as claimed in claim 17 in which the outer needles are arranged in a generally cylindrical pattern for activation by EM power to ablate the tissue therebetween for example, to form a cylindrical hollow volume of ablated tissue.

26. A device as claimed in claim 25 in which at least one of the outer needles is arranged for activation together with the central needle to ablate a closed end for the hollow cylindrical volume; preferably in which a first conducting portion of the central needle at or near the tip thereof is arranged for activation to ablate the closed end, the closed end having for example a generally disk-shaped configuration.

27. A device as claimed in claim 26 in which at least one of the outer needles is arranged for activation together with the central needle to ablate a second closed end to the hollow generally cylindrical volume spaced from the first end in order to enclose a tumor or other tissue inside an ablated enclosure of tissue; preferably in which a second conducting portion of the central needle is provided, spaced from the first conducting portion, for activation to ablate the second closed end.

28. A method of treatment comprising:
(a) deploying with a catheter a plurality of needles, the needles together defining a structure surrounding a tumor to be treated; and
(b) heating and embolizing a shell of tissue, defined by the structure, surrounding the tumor, thereby cutting off a blood supply of the tumor.

29. A method as claimed in claim 28 in which the needles are flexible, and which includes deploying the needles to take up a curved or angular form.

30. A method of treatment as claimed in claim 28, including the step of applying RF or microwave power to the needles to heat the said shell of tissue.

31. A method as claimed in claim 30 in which adjacent needles within the structure define electrodes having opposing polarities.

32. A method as claimed in claim 28 including inserting into the tumor an elongate probe which extends along a longitudinal axis of a catheter, and measuring on the probe a characteristic of the tissue within the structure.

33. A method as claimed in claim 32 when dependent upon claim 30 including inserting into the tumor an elongate probe which extends along a longitudinal axis of the catheter, and applying power to needles of the structure of a different plurality from that of the probe.

34. A method as claimed in claim 32 including the step of measuring blood pressure, impedance or temperature.

35. A method as claimed in claim 28, including measuring blood flows within the tissue, inside the structure.

36. A method as claimed in claim 28, including aspirating at least some of the tissue within the structure.

37. A method as claimed in claim 36 including the step of cutting the tissue prior to aspiration.

38. A method as claimed in claim 28 including deploying from the catheter a first plurality of needles which define a first structure and a second plurality which define a second, smaller structure within the first structure.

39. A method as claimed in claim 38 including heating and embolizing a volume of tissue between the inner and outer structures.

40. A method as claimed in claim 28 in which the shell as well as the tumor is embolized by the application of microwave energy within the structure.

41. A method as claimed in claim 39 in which microwave energy is applied to said volume of tissue between the inner and outer structures.

42. A method as claimed in claim 28 which includes providing a generally cylindrical array of needles having generally straight operative portions, and applying EM power between the needles in the array to form an ablated cylinder of tissue, around a tumor.

43. A method as claimed in claim 42 which includes providing a central needle inside the array, and applying EM power between the central needles and at least one of the needles in the cylindrical array for forming at least one closed ablated end to the cylinder of tissue, preferably both ends thereof such as when a tumor is remote from an outer surface of an organ being treated.
